(19) United States
(12) Patent Application Publication
(10) Pub. No.: US 2007/0166697 A1
(43) Pub. Date: Jul. 19, 2007

(54) SYNTHESIS AND USE OF CROSS-LINKED HYDROPHILIC HOLLOW SPHERES FOR ENCAPSULATING HYDROPHILIC CARGO

(75) Inventor: Steven M. Menchen, Fremont, CA (US)

Correspondence Address: DECHERT LLP, P.O. BOX 10004, PALO ALTO, CA, 94303, US

(73) Assignee: Applera Corporation, Foster City, CA (US)

(21) Appl. No.: 11/618,531

(22) Filed: Dec. 29, 2006

Related U.S. Application Data
(60) Provisional application No. 60/755,676, filed on Dec. 30, 2005.

(51) Int. Cl. C12Q 1/00; C08L 39/04

(52) U.S. Cl. 435/4; 525/203; 977/902

(57) ABSTRACT

Cross-linked hydrophilic nanocapsules and various compositions and methods for their preparation and use are provided.

### 1. CROSS-REFERENCE TO RELATED APPLICATIONS

This application claims the benefit under 35 U.S.C. § 119(e) of U.S. provisional application No. 60/755,676, filed Dec. 30, 2005, the disclosure of which is incorporated herein by reference in its entirety.

### 2. BACKGROUND

Assays using encapsulated reporter systems are important tools for studying and detecting analytes in biological and industrial processes. Numerous methods have been developed for encapsulating reporter systems, including the use of cross-linked nanocapsules. Typically, these methods comprise emulsifying an organic phase with an aqueous phase to yield an oil-in-water emulsion, in which the emulsion comprises a plurality of amphiphilic polymers and a reporter system that is soluble in a non-aqueous hydrophobic phase. The addition of a cross-linking agent results in the formation of a cross-linked nanocapsule encapsulating the reporter system. Although these methods are suitable for the encapsulating water insoluble reporter systems, there is still a need to find methods suitable for encapsulating water soluble reporter systems.

### 3. SUMMARY

Provided herein are cross-linked hydrophilic nanocapsules comprising water soluble reporter systems. The nanocapsules comprise hydrophilic polymers, have a cross-linked shell domain and a hydrophilic core domain. The porosity of the shell comprising the nanocapsule can be optimized to retain the water soluble reporter system, and at the same time, allow passage of a target analyte, for example, by varying the percentage of the shell domain that is cross-linked.

Generally, methods used to encapsulate reporter systems require that the reporter system be soluble in the organic phase to maintain the hydrophilic shell. For example, U.S. Pat. No. 6,393,500 describes a method for encapsulating pharmaceutically active agents using an oil-in-water-emulsion to form micelles having a permeable cross-linked poly(acrylic acid) shells and poly(caprolactone) cores. This method precludes the use of water soluble reporter systems because the water soluble reporter system remains in the aqueous continuous phase used for micelle formation.

In contrast, the cross-linked hydrophilic nanocapsules described herein are made from reverse micelles that are formed by using an inverse emulsion system comprising an organic solvent continuous phase, water, and a plurality of amphiphilic polymers. To facilitate encapsulation of a water soluble reporter system, the reporter system is dissolved in an aqueous buffer and suspended as droplets in the organic solvent continuous phase.

In some embodiments, the polymeric amphiphiles used to form the reverse micelles comprise two polymer blocks: a hydrophilic polymer block and a hydrophobic polymer block, connected via a cleavable linker moiety. The hydrophilic polymer block comprises four or more hydrophilic monomer units, which can be optionally substituted with substituents that impart hydrophilicity to the amphiphile. The hydrophobic polymer block comprises four or more hydrophobic monomer units and two or more monomer units comprising functional groups capable of cross-linking adjacent polymers to each other in the presence of a cross-linking agent. The hydrophobic monomer unit comprises one or more functional groups, which, when present, impart hydrophobicity to the amphiphile.

In some embodiments, the cross-linked hydrophilic nanocapsules comprising a water soluble reporter system are formed by emulsifying an aqueous phase with an organic phase to yield a water-in-oil emulsion, in which the emulsion comprises a plurality of amphiphilic polymers and one or more water soluble reporter systems. The amphiphiles aggregate around the aqueous droplets creating a polymeric micelle comprising a hydrophobic shell and a hydrophilic core containing the water soluble reporter system. The hydrophobic shell is cross-linked and the hydrophobic constituents are cleaved and/or modified to yield a hydrophilic shell. In some embodiments, the hydrophilic polymer block can be cleaved from the hydrophilic shell to create a hollow core.

In some embodiments, the water soluble reporter systems described herein comprise a labeled protein and a labeled surrogate analyte. The labeled protein can be contacted with the labeled surrogate analyte to form a surrogate analyte-labeled protein complex. In some embodiments, the protein comprises a fluorescent moiety such that upon displacement of the surrogate analyte by a target analyte, an increase in the fluorescence of the fluorescent moiety can be detected.

In some embodiments, the fluorescence of the labeled protein is quenched when the surrogate analyte is bound to the protein. This quenching may be accomplished by a variety of different mechanisms. In some embodiments, the protein and surrogate analyte comprise fluorescent moieties that are capable of “self-quenching” when in close proximity to each other. In other embodiments, quenching can be achieved with the aid of a quenching moiety.

In other embodiments, the cross-linked hydrophilic nanocapsules can be used to encapsulate other water soluble materials, including therapeutic agents and diagnostic agents.

In other embodiments, the encapsulated reporter systems can be used for detecting the presence or absence of analytes of interest. The analyte reporter systems comprise a labeled protein and a labeled surrogate analyte. The labeled protein can be contacted with the labeled surrogate analyte to form a surrogate analyte-labeled protein complex. In some embodiments, the protein comprises a fluorescent moiety such that upon displacement of the surrogate analyte by a target analyte, an increase in the fluorescence of the fluorescent moiety can be detected.

### 4. BRIEF DESCRIPTION OF THE FIGURES

FIG. 1 depicts a polymeric amphiphile comprising functional groups that enable the conversion of hydrophobic substituents to hydrophilic substituents, or the conversion of hydrophilic substituents to hydrophobic substituents;

FIG. 2 depicts the assembly of polymeric amphiphiles inverse emulsion conditions;

FIG. 3 depicts the cross-linking of polymeric amphiphiles using inverse emulsion conditions;

FIG. 4 depicts the conversion of a cross-linked reverse micelle to a hydrophilic nanocapsule;

FIG. 5 depicts the conversion of a cross-linked hydrophilic nanocapsule comprising two second polymer block moieties, (A—B)ₗ and [(E—F)ₙ—(G—H)ₒ] to a cross-linked hydrophilic nanocapsule comprising one polymer block moiety, [(E—F)ₙ—(G—H)ₒ];

FIG. 6 illustrates an exemplary reporter system; and,

FIGS. 7A-7C depict an exemplary method for synthesizing a cross-linked hydrophilic nanocapsule.

### 5. DETAILED DESCRIPTION

It is to be understood that both the foregoing general description and the following detailed description are exemplary and explanatory only and are not restrictive of the compositions and methods described herein. In this application, the use of the singular includes the plural unless specifically state otherwise. Also, the use of “or” means “and/or” unless state otherwise. Similarly, “comprise,” “comprises,” “comprising,” “include,” “includes” and “including” are not intended to be limiting.

5.1 Definitions

As used herein, the following terms and phrases are intended to have the following meanings:

“Amphiphilic polymer” has its standard meaning and is intended to refer to a polymer or copolymer having at least one hydrophilic domain and at least one hydrophobic domain.

“Antibody” has its standard meaning and is intended to refer to full-length as well antibody fragments, as are known in the art, including Fab, Fab₂, single chain antibodies (Fv for example), monoclonal, polyclonal, chimeric antibodies, etc., either produced by the modification of whole antibodies or those synthesized de novo using recombinant DNA technologies.

“Detect” and “detection” have their standard meaning, and are intended to encompass detection, measurement, and characterization of an analyte.

“Reverse micelle” has its standard meaning and is intended to refer to an aggregate formed by amphipathic molecules in an organic continuous phase such that their nonpolar ends or portions are in contact with the organic phase and their polar ends or portions are in the interior of the aggregate. A reverse micelle can take any shape or form, including but not limited to, spheres, cylinders, discs, needles, cones, vesicles, globules, rods, ellipsoids, and any other shape that a reverse micelle can assume under the conditions described herein, or any other shape that can be adopted through aggregation of the amphiphilic polymers.

“Protein” has its standard meaning and is intended to refer to proteins, oligopeptides and peptides, derivatives and analogs, including proteins containing non-naturally occurring amino acids and amino acid analogs, and peptidomimetic structures, and includes proteins made using recombinant techniques, i. e. through the expression of a recombinant nucleic acid.

“Quench” has its standard meaning and is intended to refer to a reduction in the fluorescence intensity of a fluorescent group or moiety as measured at a specified wavelength, regardless of the mechanism by which the reduction is achieved. As specific examples, the quenching can be due to molecular collision, energy transfer such as FRET, photoinduced electron transfer such as PET, a change in the fluorescence spectrum (color) of the fluorescent group or moiety or any other mechanism (or combination of mechanisms). The amount of the reduction is not critical and can vary over a broad range. The only requirement is that the reduction be detectable by the detection system being used. Thus, a fluorescence signal is “quenched” if its intensity at a specified wavelength is reduced by any measurable amount. A fluorescence signal is “substantially quenched” if its intensity at a specified wavelength is reduced by at least 50%, for example by 50%, 60%, 70%, 75%, 80%, 85%, 90%, 95%, 96%, 97%, 98%, 99% or even 100%.

5.2 Cross-Linked Hydrophilic Nanocapsules

The present disclosure provides cross-linked hydrophilic nanocapsules that can encapsulate a wide variety of water soluble reporter systems and agents (e.g., therapeutic agents, diagnostic agents, etc.). In some embodiments, the nanocapsules described herein can be used to deliver the encapsulated reporter systems and agents to cells. The cross-linked hydrophilic nanocapsules are formed under inverse emulsion conditions in which amphiphilic polymers are added to an organic solvent comprising a water soluble reporter system or agent dissolved in aqueous droplets. The amphiphilic polymers assemble around the aqueous droplets creating a reverse polymeric micelle comprising a hydrophobic shell and a hydrophilic core comprising the water soluble reporter system(s). Addition of a cross-linking agent results in the formation of a cross-linked hydrophobic shell. Removal or modification of functional groups imparting water insolubility to the amphiphile creates a cross-linked hydrophilic shell surrounding a hydrophilic core.

5.3 Polymeric Amphiphiles

Amphiphilic polymers useful in the compositions and methods described herein can be synthesized from polymer blocks comprising monomers with various functionalities. As used herein, “polymer block” or “block” refers to a region or segment along the backbone of a polymer which is characterized by similar hydrophilicity, hydrophobicity, or other chemistry, such as functional groups and/or substituents which are capable of promoting co-polymerization or forming covalent bonds with cross-linking agents. The exact number and/or composition of the polymer blocks can be selectively varied. For example, in some embodiments, the amphiphilic polymers comprise two blocks, one hydrophilic and one hydrophobic block. In other embodiments, the amphiphilic polymers can comprise three, four or more blocks. In embodiments employing three or more blocks, the combination of hydrophilic and hydrophobic blocks can be varied provided that the resulting amphiphilic polymer has sufficient hydrophobic and hydrophilic character to form a reverse micelle.

The various blocks comprising an amphiphilic polymer can be connected directly or indirectly through a linker moiety. In some embodiments, a linker moiety is used to attach the blocks to each other. Linker moieties can be selected to form permanent linkages or temporary linkages depending on the application. For example, if nanocapsules comprising hollow cores are desired, a cleavable linker moiety can be used to attach the blocks to each other.

Typically, each block comprises one, two, three, four, or more monomers that can be connected directly or indirectly through a linker moiety. The exact number and/or composition of the monomers can be selectively varied. In embodiments employing two or more monomers, each monomer can be the same, or some or all of the monomers can differ.

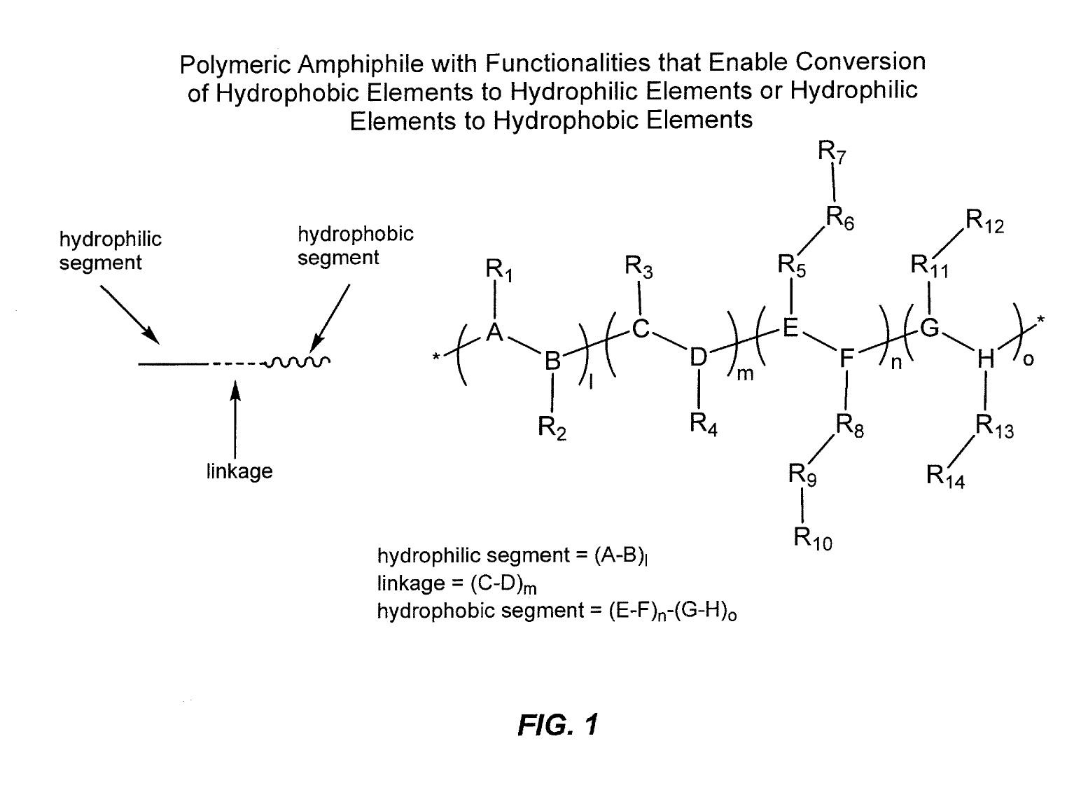

In some embodiments, the polymeric amphiphiles are synthesized from hydrophilic and hydrophobic blocks. FIG. 1 illustrates an exemplary embodiment of a amphiphilic polymer that can be used as described herein. As illustrated in FIG. 1, the amphiphilic polymer generally comprises a hydrophilic block (represented by (A—B)ₗ), a hydrophobic block (represented by (E—F)ₙ—(G—H)ₒ) and a linker moiety (represented by (C—D)ₘ). The hydrophilic block and the linker moiety can be optionally substituted with one or more substituents (represented by R₁, R₂, R₃, and R₄) that can impart additional characteristics. For example, hydrophilic block (A—B)ₗ) can be substituted with R₁ and/or R₂ which include substituents that are capable of imparting water solubility to hydrophilic block (A—B)ₗ).

As depicted in FIG. 1, hydrophobic block (E—F)ₙ—(G—H)ₒ can comprise one or more functional groups: R₅—R₆—R₇ represent a first functional group, R₈—R₉—R₁₀ represent a second functional group, R₁₁—R₁₂ represent a third functional group, and R₁₃—R₁₄ represent a fourth functional group. The functional groups impart desirable characteristics, such as, promoting polymerization, providing reactive groups that can react with cross-linking agents, or imparting water soluble or water insoluble characteristics to the polymer.

Suitable hydrophilic and hydrophobic blocks for use in the compositions and methods described herein are described below.

5.3.1 Hydrophilic Blocks

As depicted in FIG. 1, the hydrophilic block comprises a monomer unit represented by (A—B)ₗ that imparts water solubility to the polymer block. The number of monomer units (represented by l) comprising the hydrophilic block can be selected provided that the resulting block has sufficient hydrophilic character to integrate the resultant amphiphilic polymer into a reverse micelle. In some embodiments, the hydrophilic block comprises from 4 to 8 monomers, or from 4 to 12 monomers, or from 4 to 16 monomers, or from 4 to 20 monomers, or from 6 to 10 monomers, or from 6 to 14 monomers, or from 6 to 18 monomers or from 6 to 20 monomers. Exemplary hydrophilic blocks comprise 4, 5, 6, 8, 9, 10, 11, 12, 13, 14, 15, 16, 17, 18, 19, or 20 monomers.

The monomers comprising the hydrophilic block can be attached directly or indirectly via a linkage moiety. In some embodiments, the monomers are attached directly via atoms and linkages contributed by the terminus of each monomer comprising the hydrophilic block. For example, the terminus of each monomer can comprise complementary reactive groups capable of forming covalent linkages with one another. Pairs of complementary groups capable of forming covalent linkages are well known. In some embodiments, one terminus comprises a nucleophilic group and the other terminus comprises an electrophilic group. “Complementary” nucleophilic and electrophilic groups (or precursors thereof that can be suitable activated) useful for effecting linkages stable to biological and other assay conditions are well known. Examples of suitable complementary nucleophilic and electrophilic groups, as well as the resultant linkages formed therefrom are provided in Table 1.

TABLE 1

| Electrophilic Group | Nucleophilic Group | Resultant Covalent Linkage |
|---|---|---|
| activated esters* | amines/anilines | carboxamides |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| Alkyl halides | amines/anilines | alkyl amines |
| Alkyl halides | carboxylic acids | esters |
| Alkyl halides | thiols | thioethers |
| Alkyl halides | alcohols/phenols | ethers |
| Alkyl sulfonates | thiols | thioethers |
| Alkyl sulfonates | carboxylic acids | esters |
| Alkyl sulfonates | alcohols/phenols | esters |
| anhydrides | alcohols/phenols | esters |
| anhydrides | amines/anilines | caroboxamides |
| aryl halides | thiols | thiophenols |
| aryl halides | amines | aryl amines |
| aziridines | thiols | thioethers |
| boronates | glycols | boronate esters |
| carboxylic acids | amines/anilines | carboxamides |
| carboxylic acids | alcohols | esters |
| carboxylic acids | hydrazines | hydrazides |
| carbodiimides | carboxylic acids | N-acylureas or anhydrides |
| diazoalkanes | carboxylic acids | esters |
| epoxides | thiols | thioethers |
| haloacetamides | thiols | thioethers |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | Thiols | thioethers |
| phosphoramidites | Alcohols | phosphate esters |
| silyl halides | Alcohols | silyl ethers |
| sulfonate esters | amines/anilines | alkyl amines |
| sulfonate esters | Thiols | thioethers |
| sulfonate esters | carboxylic acids | esters |
| sulfonate esters | Alcohols | esters |
| sulfonyl halides | amines/anilines | sulfonamides |
| sulfonyl halides | phenols/alcohols | sulfonate esters |
| Diazonium salt | aryl | azo |

*Activated esters, as understood in the art, generally have the formula —C(O)Z, where Z is a good leaving group (e.g., oxysuccinimidyl, oxysulfosuccinimidyl, 1-oxybenzotriazolyl, etc.).
**Acyl azides can rearrange to isocyanates.

In some embodiments, A and B together represent a group of atoms that contribute to the water solubility or hydrophilicity of the hydrophilic block. Exemplary (A—B) monomers comprise —O—CH₂—CH₂—, NH—CH₂—CH₂—, and/or —S(O)—CH₂—CH₂—.

In some embodiments, A and B together represent a group of atoms, none of which contribute to the water solubility of the hydrophilic block. In these embodiments, A and/or B are substituted with at least one substituent, represented by R₁ and R₂ in FIG. 1, which imparts water solubility to the hydrophilic block. In an exemplary embodiment, A and B together are —CH—CH₂— and R₁ and/or R₂ can be —C(O)NH₂, —C(O)OH, —C(O)O⁻, SO₃⁻, or a combination thereof. 5.3.2 Hydrophobic Block

As depicted in FIG. 1, the hydrophobic block typically comprises two different monomers (E—F)ₙ and (G—H)ₒ. The monomer represented by (E—F)ₙ imparts water insolubility to the hydrophobic block, either alone, or in the presence of one or both of the functional groups represented by R₅—R₆—R₇ and R₈—R₉—R₁₀. The monomer represented by (G—H)ₒ, either alone, or in the presence of one or both of the functional groups represented by R₁₁—R₁₂ and R₁₃—R₁₄ provides reactive groups that impart physical or chemical cross-linking potential to the hydrophobic block in the presence of a cross-linking agent.

In some embodiments, E and F together comprise a group of atoms that are hydrophilic. In these embodiments, E and F comprise at least one functional group, represented by R₅—R₆—R₇ and/or R₈—R₉—R₁₀, that imparts water insolubility to the hydrophobic block. Water insolubility can be contributed by one member (represented by a single “R” substituent), two members (represented by two “R” substituents), or all members (represented by all “R” substituents comprising a functional group) of the functional group. For example, in some embodiments, all members of the functional group comprise “R” substituents that contribute water insolubility to the hydrophobic block. In these embodiments, the functional group can be removed to yield a water soluble block, (E—F)ₙ —(G—H)ₒ. Chemical or physical methods can be used to remove the functional groups that impart water insolubility to yield a water soluble block comprising (E—F)ₙ —(G—H)ₒ. Agents suitable for removing functional groups, include, but are not limited to, chemical cleavage agents such as hydroxide, acid, fluoride and amines, enzymatic cleavage agents, such as esterases, and physical agents, such as light.

In some embodiments, E and F together comprise a group of atoms that are not soluble in water. In these embodiments, at least one or more of the functional groups represented by R₅—R₆—R₇ and/or R₈—R₉—R₁₀, comprise one or more R substituents that impart water insolubility to the hydrophobic block comprising (E—F)ₙ —(G—H)ₒ when present. These R substituents can be removed to yield water soluble constituents. For example, if the functional group represented by R₅—R₆—R₇ is present and R₇ comprises a water insoluble group, the removal of R₇ yields a water soluble functional group represented by R₅—R₆, that is capable of imparting water solubility to the block comprising (E—F)ₙ—(G—H)ₒ. In another example, if the functional group represented by R₈—R₉—R₁₀ is present and R₁₀ comprises a water insoluble group, the removal of R₁₀ yields a water soluble functional group represented by R₈—R₉, that is capable of imparting water solubility to the block comprising (E—F)ₙ—(G—H)ₒ. In another example, if both functional groups are present, R₅—R₆—R₇ and R₈—R₉—R₁₀, R₇ and/or R₁₀ can comprise water insoluble groups, which upon removal yield water soluble functional groups represented by R₅—R₆, and R₈—R₉, that are capable of imparting water solubility to the block comprising (E—F)ₙ—(G—H)ₒ.

In addition to the hydrophobicity characteristics imparted by R₇ and/or R₁₀, other functional characteristics can be provided by R₅, R₆, R₈, and R₉. For example, in some embodiments R₅ and/or R₈ can comprise substituents that enable polymerization of the one or more (E—F) monomers. In another example, R₆ and/or R₉ can comprise linkage moieties that connect R₅ and R₇ and R₈ and R₁₀.

In some embodiments, at least one of the “R” substituents comprising functional groups R₅, R₆, and R₇ and R₈, R₉, and R₁₀, is not hydrogen.

In some embodiments, (E—F) together are —CH(R₅—R₆—R₇)—CH(R₈—R₉—R₁₀), R₅ and/or or R₈ is carbonyl, R₆ and/or R₉ is selected from oxygen and nitrogen, and R₇ and/or R₁₀ is selected from an alkyl containing from 4 to 20 carbon atoms, phenyl, benzyl or trialkylsilyl.

In some embodiments, (E—F) together are —CH(R₅—R₆—R₇)—CH(R₈—R₉—R₁₀), R₅ and/or or R₈ is oxygen, R₆ and/or R₉ comprises carbonyl, and R₇ and/or R₁₀ is selected from an alkoxy containing from 3 to 10 carbon atoms, phenoxy, benzoxy and trialkylsiloxy.

In some embodiments, (E—F) together are —CH(R₅—R₆—R₇)—CH(R₈—R₉—R₁₀), R₅ and/or or R₈ is selected from nitrogen or an amine, R₆ and/or R₉ is carbonyl, and R₇ and/or R₁₀ is selected from an alkoxy containing from 3 to 10 carbon atoms, phenoxy, benzoxy and trialkylsiloxy.

In some embodiments, (E—F) together are —CH(R₅—R₆—R₇)—CH(R₈—R₉—R₁₀), R₅ and/or or R₈ is selected from sulfate, phosphate or borate, and R₇ and/or R₁₀ is selected from an alkyl containing from 4 to 20 carbon atoms, phenyl, benzyl or trialkylsilyl.

In some embodiments, (E—F) together are —CH(R₅—R₆—R₇)—CH(R₈—R₉—R₁₀), R₅ and/or or R₈ is carbonyl, and R₇ is selected from an alkoxy containing from 3 to 10 carbon atoms, phenoxy, benzoxy and trialkylsiloxy.

In some embodiments, (E—F) together are —CH₂—CH₂—N(R₅—R₇), R₅ is carbonyl, and R₇ is selected from an alkoxy containing from 3 to 10 carbon atoms, phenoxy, benzoxy and trialkylsiloxy.

In other embodiments, (E—F) together are —CH₂—CH—N(R₅—R₇), R₅ is carbonyl, and R₇ is selected from an alkoxy containing from 3 to 10 carbon atoms, phenoxy, benzoxy and trialkylsiloxy.

In other embodiments, (E—F) together are —CH₂—CH—O(R₅—R₇), R₅ is carbonyl, and R₇ is selected from an alkoxy containing from 3 to 10 carbon atoms, phenoxy, benzoxy and trialkylsiloxy.

The number of monomer units (represented by n) comprising monomer (E—F) can be selected provided that the resulting block has sufficient hydrophobic character to integrate the resultant amphiphilic polymer into a reverse micelle. In some embodiments, the hydrophobic block comprises from 4 to 8 (E—F) monomers, or from 4 to 12 (E—F) monomers, or from 4 to 16 (E—F) monomers, or from 4 to 20 (E—F) monomers, or from 6 to 10 (E—F) monomers, or from 6 to 14 (E—F) monomers, or from 6 to 18 (E—F) monomers or from 6 to 20 (E—F) monomers. Exemplary hydrophobic blocks comprise 4, 5, 6, 8, 9, 10, 11, 12, 13, 14, 15, 16, 17, 18, 19, or 20(E—F)monomers.

The (E—F) monomers can be attached directly, or indirectly via a linkage moiety as described above for the (A—B) monomers.

In some embodiments, the (G—H) monomer comprises at least one functional group represented by R₁₁—R₁₂ and/or R₁₃—R₁₄, that provides reactive groups that impart physical or chemical cross-linking potential to the hydrophobic block in the presence of a cross-linking agent. (G—H) can comprise a multiple atom unit in which none of the atoms contains cross-linking potential or a multiple atom unit in which one or more of the atoms contains cross-linking potential.

Cross-linker reactivity, specificity, and solubility characteristics are well known in the art. Guidance for selecting appropriate cross-linking agents can be found in Mattson et al., MOL BIOL REP. Apr; 17(3):167-83 (1993), and Double-Agents™ Cross-linking Reagents, Selection Guide, Pierce Biotechnology Inc., 2003). See also, Wong, 1993, Chemistry of Protein Conjugation and Cross-linking, CRC Press, Boca Raton.

Functional groups R₁₁—R₁₂ and R₁₃—R₁₄ can comprise one or more substituents that are reactive with a cross-linking agent. For example, in some embodiments, R₁₁—R₁₂ and R₁₃—R₁₄ can comprise electrophilic reactive groups that can be cross-linked using nucleophilic agents. In other embodiments, R₁₁—R₁₂ and R₁₃—R₁₄ can comprise nucleophilic reactive groups that can be cross-linked using electrophilic agents. Examples of suitable complementary nucleophilic and electrophilic groups, as well as the resultant linkages formed therefrom, are provided in Table 1 above.

In some embodiments, R₁₁—R₁₂ and/or R₁₃—R₁₄ comprise electrophilic moieties. In these embodiments, suitable cross-linking agents include, but are not limited to, nucleophilic agents with at least two nucleophilic functional groups, such as polyamines (e.g., ethylene diamine), polyhydroxols (e.g., ethylene glycol), and polysulfides (e.g., ethylene disulfide).

In some embodiments, R₁₁—R₁₂ and/or R₁₃—R₁₄ comprise nucleophilic moieties. In these embodiments, suitable cross-linking agents include, but are not limited to, electrophilic agents with at least two electrophilic functional groups, such as polyacid chlorides (e.g., adipoyl chloride), electrophilic agents comprising moieties with multiple Michael acceptors (e.g., 1,2-bismaleimidoethane), polyhydrocarbons (e.g., ethylene dibromide), polyisocyanates (e.g., toluene diisocyante), and polyesters (e.g., bis-N-hydroxysuccinimidyl adipate).

In some embodiments, R₁₁—R₁₂ and R₁₃—R₁₄ can comprise latent anionic species that can be cross-linked with multivalent metal cations.

Additional examples of suitable activatable reactive moieties are described in U.S. patent application Ser. No. 11/375,825, filed Mar. 15, 2006, entitled “The Use of Antibody-Surrogate Antigen Systems for Detection of Analytes,” incorporated herein by reference in its entirety, including, but not limited to, molecules that can be activated by light, pH, heat, or electrochemically.

In addition to reactive groups comprising cross-linking potential, other functional characteristics can be provided by R₁₁, R₁₂, R₁₃, and R₁₄. For example, in some embodiments, R₁₁and/or R₁₃ can comprise substituents that enable co-polymerization of two or more (G—H) monomers with four or more (E—F) monomers, and R₁₂ and/or R₁₄ can comprise reactive functional substituents or groups that form covalent bonds with a cross-linking agent, or form physical cross-linking bonds (e.g., ionic bonds) to reactive R₁₂ and/or R₁₄ groups on adjacent amphiphiles. In another example, R₁₁and/or R₁₃ can comprise substituents that enable co-polymerization and substituents that impart reactivity to cross-linking agents.

In some embodiments, at least one of the “R” substituents comprising functional groups R₅, R₆, and R₇ and R₈, R₉, and R₁₀, is not hydrogen.

In some embodiments, (G—H) together comprises —CH(R₁₁—R₁₂)—CH(R₁₃—R₁₄)—, wherein R₁₁ and/or R₁₃ is selected from carbonyl, sulfate, phosphate, or borate, and R₁₂ and/or R₁₄ is an electrophilic substituent selected from alkoxy, phenoxy, substituted phenoxy, halogen, N-hydroxysuccinimidyl, and organic and inorganic mixed anhydrides, such as acetate and phosphate.

In some embodiments, (G—H) together comprises —CH(R₁₁—R₁₂)—CH(R₁₃—R₁₄)—, wherein R₁₁ and/or R₁₃ is selected from carbonyl, sulfate, phosphate, or borate, and R₁₂ and/or R₁₄ is an electrophilic substituent comprising a Michael acceptor, such as vinyl.

In some embodiments, (G—H) together comprises —CH(R₁₁—R₁₂)—CH(R₁₃—R₁₄)—, wherein R₁₁ and/or R₁₃ is selected from oxygen or amine, and R₁₂ and/or R₁₄ is an electrophilic substituent selected from alkoxycarbonyl, phenoxycarbonyl, and halocarbonyl.

In some embodiments, (G—H) together comprises —CH(R₁₁—R₁₂)—CH(R₁₃—R₁₄)—, wherein R₁₁ and/or R₁₃ is selected from oxygen or amine, and R₁₂ and/or R₁₄ is an electrophilic substituent comprising a Michael acceptor selected from maleimide, vinylcarbonyl, alkynylcarbonyl, vinylsulfone, and alkynyl sulfone.

In some embodiments, (G—H) together comprises —CH(R₁₁—R₁₂)—CH(R₁₃—R₁₄)—, wherein R₁₁ and/or R₁₃ is carbonyl, and R₁₂ and/or R₁₄ is a nucleophilic substituent selected from alcohol, polyol, amine, polyamine, sulfide or polysulfide.

The number of monomer units (represented by o) comprising monomer (G—H) can be selected provided that the resulting block has sufficient cross-linking potential to form a cross-linked hydrophilic nanocapsule with the desired porosity. In some embodiments, the hydrophobic block comprises from 2 to 6 (G—H) monomers, or from 3 to 6 (G—H) monomers, or from 4 to 6 (G—H) monomers. Exemplary hydrophobic blocks comprise 2, 3, 4, 5, or 6 (G—H) monomers.

The (G—H) monomers can be attached directly, or indirectly via a linkage moiety as described above for the (A—B) monomers.

5.3.3 Linker Moieties

As depicted in FIG. 1, (C—D)ₘ comprises a linker moiety for attaching a hydrophilic block (A—B)ₗ to a hydrophobic block (E—F)ₙ—(G—H)ₒ. Typically, (C—D) comprises at least one atom that can form a covalent attachment with at least one atom at the terminus of a hydrophilic block and at least one atom that can form a covalent attachment with at least one atom at the terminus of a hydrophobic block. The number of moieties (represented by o) can be 0 or 1.

The linker moiety can be selected to have specified properties. For example, the linker moiety can be hydrophobic in character, hydrophilic in character, long or short, rigid, semirigid or flexible, permanent or labile, depending upon the particular application. The linker moiety can be optionally substituted with one or more substituents, e.g., R₃ and/or R₄, which can be the same or different, thereby enhancing the linking chemistry of (C—D). In some embodiments, the optional substituents in combination with (C—D) can form a “polyvalent” linking moiety capable of conjugating or linking additional molecules or substances to the amphiphile. In certain embodiments, however, the linker moiety does not comprise such additional substituents or linking groups.

A wide variety of linker moieties comprised of stable bonds are known in the art, and include by way of example and not limitation, alkyldiyls, substituted alkyldiyls, alkylenos (e.g., alkanos), substituted alkylenos, heteroalkyldiyls, substituted heteroalkyldiyls, heteroalkylenos, substituted heteroalkylenos, acyclic heteroatomic bridges, aryldiyls, substituted aryldiyls, arylaryldiyls, substituted arylaryldiyls, arylalkyldiyls, substituted arylalkyldiyls, heteroaryldiyls, substituted heteroaryldiyls, heteroaryl-heteroaryl diyls, substituted heteroaryl-heteroaryl diyls, heteroarylalkyldiyls, substituted heteroarylalkyldiyls, heteroaryl-heteroalkyldiyls, substituted heteroaryl-heteroalkyldiyls, and the like. Thus, a linker moiety can include single, double, triple or aromatic carbon-carbon bonds, nitrogen-nitrogen bonds, carbon-nitrogen bonds, carbon-oxygen bonds, carbon-sulfur bonds, silicon-oxygen bonds, silicon-carbon bonds, and combinations of such bonds, and may therefore include functionalities such as carbonyls, ethers, thioethers, carboxamides, sulfonamides, ureas, urethanes, hydrazines, etc. In some embodiments, the linker moiety has from 1-20 non-hydrogen atoms selected from the group consisting of C, N, O, P, and S and is composed of any combination of ether, thioether, amine, ester, carboxamide, sulfonamides, hydrazide, aromatic and heteroaromatic groups.

Choosing a linker moiety having properties suitable for a particular application is within the capabilities of those having skill in the art. For example, if a rigid linker moiety is desired, the linker moiety may comprise a rigid polypeptide such as polyproline, a rigid polyunsaturated alkyldiyl or an aryldiyl, biaryldiyl, arylarydiyl, arylalkyldiyl, heteroaryldiyl, biheteroaryldiyl, heteroarylalkyldiyl, heteroaryl-heteroaryldiyl, etc. Where a flexible linker moiety is desired, the linker moiety may comprise a flexible polypeptide such as polyglycine or a flexible saturated alkanyldiyl or heteroalkanyldiyl. Hydrophilic linker moieties may comprise, for example, polyalcohols, polyethers, such as polyalkyleneglycols, or polyelectroyles, such as polyquatemary amines. Hydrophobic linker moieties may comprise, for example, alkyldiyls or aryldiyls.

In some embodiments, the linker moiety comprises a peptide bond.

In some embodiments, the linker moiety formed by (C—D) is a labile linker. For example, in some embodiments, C and D together are silyl ether. In this embodiment, the addition of fluoride or an acid can be used to cleave the linkage formed between C and D.

In some embodiments, C and D together are an ester. In this embodiment, the addition of an acid or base can be used to cleave the linkage formed between C and D.

In some embodiments, C and D together are an imine. In this embodiment, the addition of an acid or base can be used to cleave the linkage formed between C and D.

In some embodiments, C and D together are an olefin. In this embodiment, the addition of permanganate, chromate or ozone can be used to cleave the linkage formed between C and D.

In some embodiments, C and D together are an anyhdride. In this embodiment, the addition of an acid or base can be used to cleave the linkage formed between C and D.

In some embodiments, C and D together are an acetal. In this embodiment, the addition of an acid can be used to cleave the linkage formed between C and D.

5.3.4 Methods of Making Cross-Linked Hydrophilic Nanocapsules

Methods for synthesizing amphiphilic polymers are well known in the art. An exemplary method for synthesizing amphiphilic polymers for use in the methods and compositions described herein is described in Example 1.

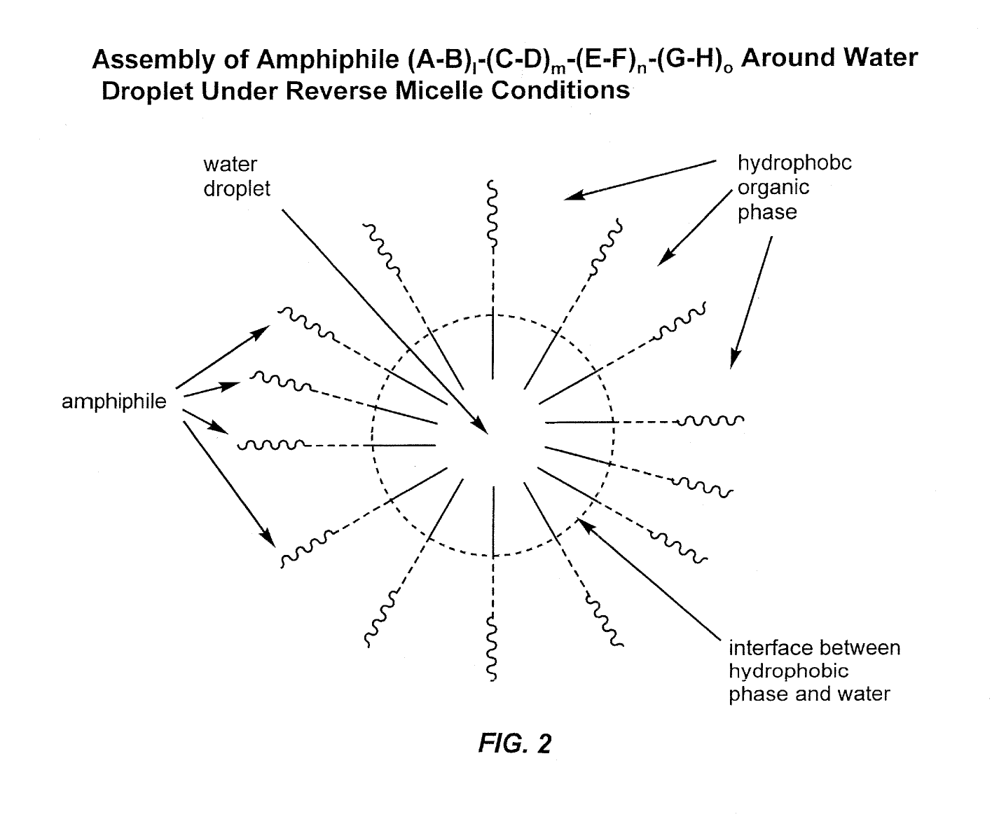

Cross-linked hydrophilic nanocapsules encapsulating one or more water soluble reporter systems or agents can be formed by suspending the amphiphilic polymers in an organic suspension of aqueous droplets comprising the water soluble reporter systems. An exemplary method for forming cross-linked hydrophilic nanocapsules is depicted in FIGS. 2-5. FIG. 2 illustrates the assembly of amphiphilic polymers around aqueous droplets to create a reverse micelle comprising a hydrophobic shell and a hydrophilic core. In the absence of a cross-linking agent, polymeric amphiphiles comprising (A—B)ₗ)—(C—D)ₘ—(E—F)ₙ—(G—H)ₒ) are soluble in organic solvents, such as those described below.

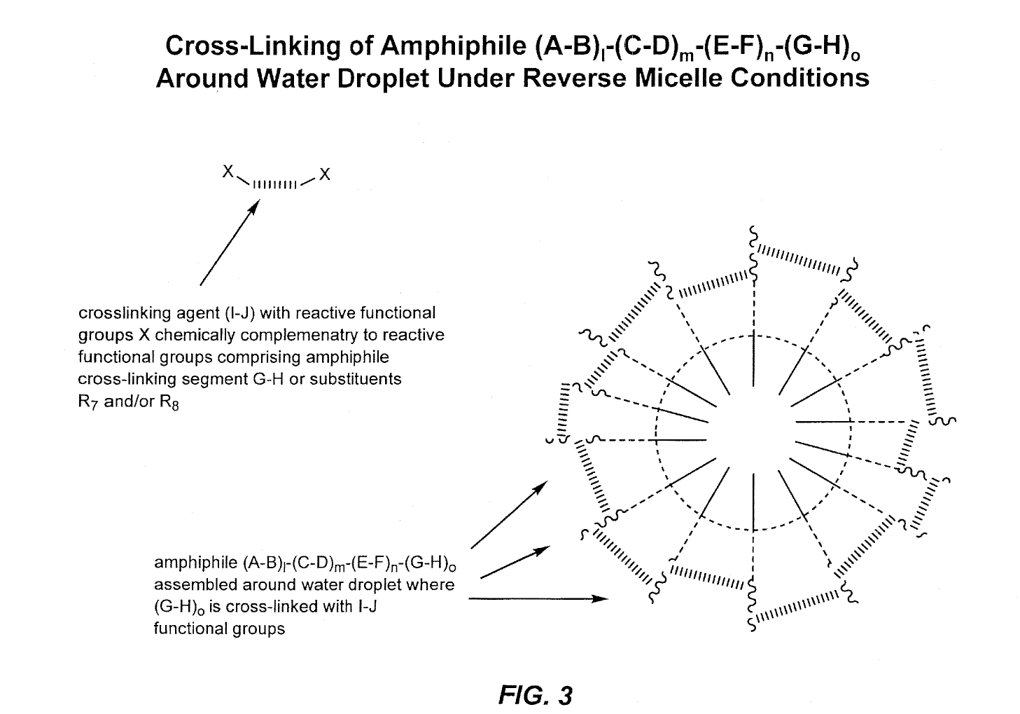

As illustrated in FIG. 3, the addition of a cross-linking agent results in the formation of a cross-linked hydrophobic shell. In the presence of the cross-linking agent, polymeric amphiphiles comprising (A—B)ₗ)—(C—D)ₘ—(E—F)ₙ—(G—H)ₒ) form insoluble linkages to adjacent polymeric amphiphiles comprising (A—B)ₗ)—(C—D)ₘ—(E—F)_{—(G—H)}ₒ) through R₁₁—R₁₂ and/or R₁₃—R₁₄.

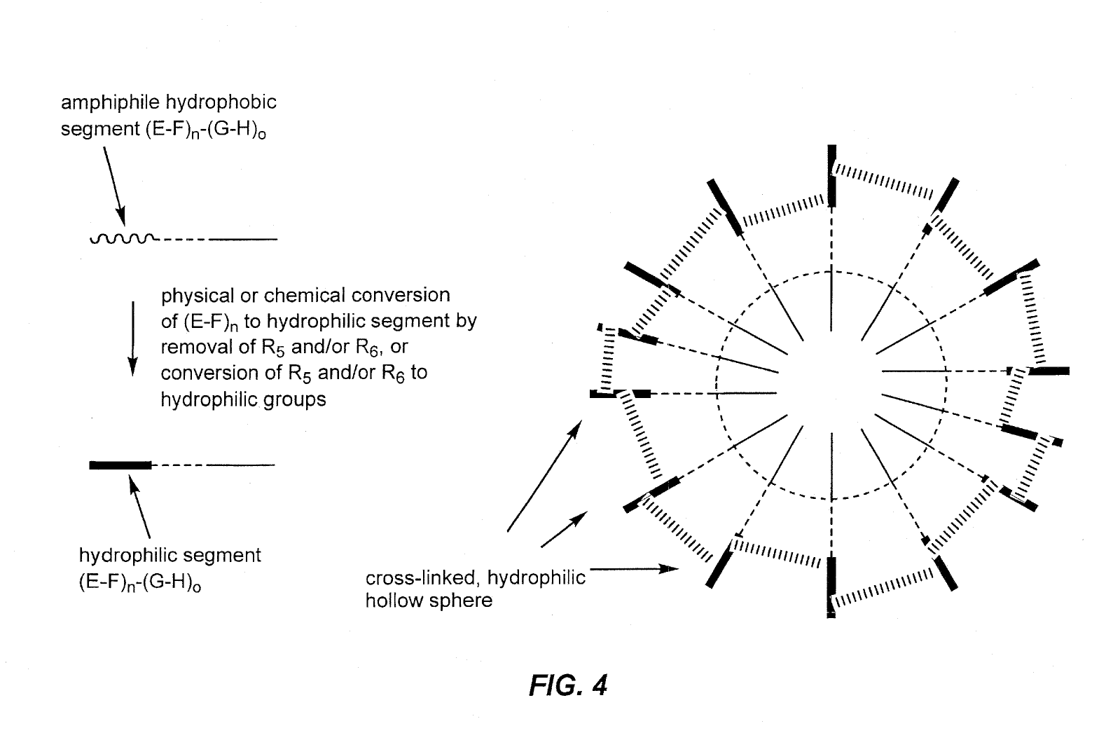

As depicted in FIG. 4, removal or modification of the functional groups imparting hydrophobicity to the hydrophobic blocks creates a cross-linked hydrophilic shell.

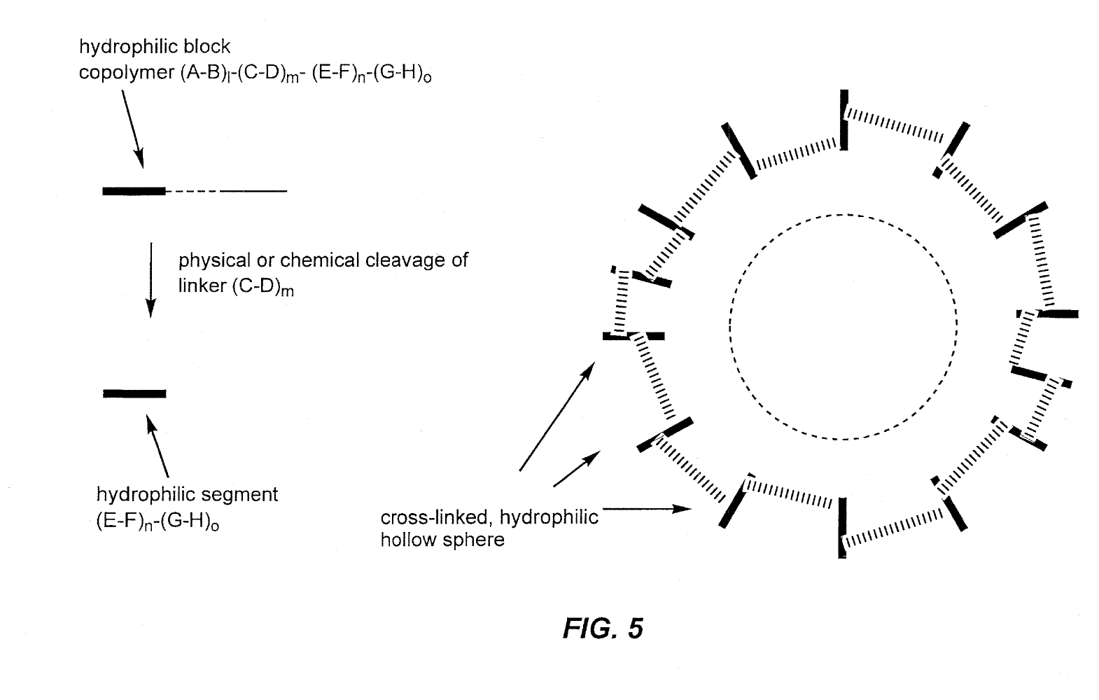

As depicted in FIG. 5, in some embodiments, the core hydrophilic blocks can be cleaved leaving a hollow sphere comprising a water soluble reporter system or agent.

Methods for making inverse emulsions, e.g., water-in-oil emulsions, are well known in the art. Guidance for selecting appropriate conditions for forming reverse micelles using water-in-oil emulsions can be found in Barton and Capek, 1994, in “Radical Polymerization in Disperse Systems,” pages 186-210, Ellis Horwood.

In some embodiments, in the presence of organic suspensions of aqueous solutions the amphiphilic polymers can self-assemble by adding them at an appropriate concentration in an organic aqueous solvent system effective in orienting the amphiphilic polymers into reverse micelles. The appropriate concentration of amphiphilic polymers and aqueous phase can be determined empirically. Alternatively, active processes such as applying energy via heating, sonication, shearing can be used to aid in orienting the amphiphilic polymers into reverse micelles.

Suitable organic solvents for use in the methods described herein include, but are not limited to, oil (e.g., paraffin oil), chlorinated hydrocarbons (e.g., carbon tetrachloride, chlorotoluene, dichlorobenzene) and aromatic hydrocarbons (e.g., benzene, ethyl benzene, naphthalene, nitrobenzene, tetrahydrofuran, and xylene).

Suitable aqueous solvents include, but are not limited to water.

The nanocapsules and reverse micelles described herein can assume a variety of shapes, including spheres, cylinders, discs, needles, cones, vesicles, globules, rods, ellipsoids, and any other shape that can be adopted through the aggregation of the amphiphilic polymers.

The size of the nanocapsules can be larger than a micron, or less than a micron. For example, in embodiments comprising spherical or mostly spherical nanocapsules, the nanocapsules can have a mean diameter from about 2 nm to about 1000 nm, from about 5 nm to about 200 nm, from about 10 nm to about 100 nm.

The thickness of the cross-linked shell of the nanocapsules can be in the range from about 0.5 nm to about 50 nm, from about 1 nm to 25 nm and from about 3 nm to about 10 nm.

In some embodiments, the cross-linked shell can comprise neutral or charged groups. For example, as described in Example 1, if a significant percentage of the backbone of the hydrophobic polymer block comprises trimethylsilyl (TMS) ethers of the polymerizable monomer hydroxyethyl methacrylate (HEMA), a neutral shell is formed. In another example, if the TMS ester of methacrylic acid is used in place of trimethylsilyl, an anionic shell can be formed.

The porosity of the nanocapsules can be controlled in a number of ways, such as by varying the number of (G—H) monomers comprising the hydrophobic blocks, by varying the structure of the cross-linking substituents utilized in the compositions and methods described herein, by varying the chemical composition of the monomers comprising the hydrophobic block, by adding small amounts of amphiphiles that lack cross-linking substituents during reverse micelle formation, and combinations thereof. Thus, depending on the application, the nanocapsules used to encapsulate the reporter complexes can be permeable, semi-permeable or impermeable.

In some embodiments, the porosity of the shell comprising the nanocapsule used to encapsulate the reporter system is selected to retain the reporter system, and at the same time, allow passage of the target analyte. The porosity of the particle membrane is such that it allows passage of elements that are less than or equal to 0.5 nm to 5.0 nm in diameter. In some embodiments, the pore diameter of the particles is less than or equal to 5.0 nm. In some embodiments, the pore diameter of the particles is less than or equal to 2.0 nm. In some embodiments, the pore diameter of the particles is less than or equal to 1.5 nm. In some embodiments, the pore diameter of the particles is less than or equal to 1.0 nm. In some embodiments, the pore diameter of the particles is less than or equal to or equal to 0.5 nm.

In some embodiments, a targeting moiety can be attached to the nanocapsule and used, for example, to target the nanocapsule to a particular cell or collection of cells. As used herein, “targeting moiety” includes any chemical moiety capable of binding to, or otherwise transporting through, a particular type of membrane and/or organelle in a cell, tissue, or organ. A variety of agents that direct compositions to particular cells are known in the art (see, for example, Cotten et al., Methods Enzym, 217: 618, 1993), and U.S. Pat. Nos. 6,692,911 and 6,835,393). Suitable non-limiting examples of targeting moieties include proteins (such as insulin, EGF, or transferrin), lectins, antibodies and fragments, carbohydrates, lipids, oligonucleotides, DNA, RNA, or small molecules and drugs. Additional examples, of useful targeting moieties include, but are in no way limited to, transfection agents such as Pro-Ject (Pierce Biotechnology), viral peptide fragments such as transportans, pore forming toxins such as streptolysin-O, hydrophobic esters, polycations such as polylysine, asiaglycoproteins, and diphtheria toxin.

5.4 Methods for Using Encapsulated Reporter Systems

Also provided herein are assays for detecting the presence or absence of a target analyte in a sample. The sample to be tested can be any suitable sample selected by the user. The sample can be naturally occurring or man-made. For example, the sample can be a blood sample, tissue sample, cell sample, buccal sample, skin sample, urine sample, water sample, or soil sample. The sample can be from a living organism, such as a eukaryote, prokaryote, mammal, human, yeast, or bacterium. The sample can be a cell, tissue, or organ. The sample can be processed prior to contact with a surrogate analyte-protein complex or labeled protein of the present teachings by any method known in the art. For example, the sample can be subjected to a lysing step, precipitation step, column chromatography step, heat step, etc.

The assays comprise contacting a sample with a “reporter system” comprising a surrogate analyte-protein complex or a labeled protein as described in U.S. application entitled “The Use of Antibody-Surrogate Antigen Systems for Detection of Analytes,” Ser. No. 60/622,412, filed on Mar. 15, 2005, and U.S. utility application Ser. No. 11/375,825, filed Mar. 15, 2006, the disclosures of which are incorporated herein by reference in their entireties.

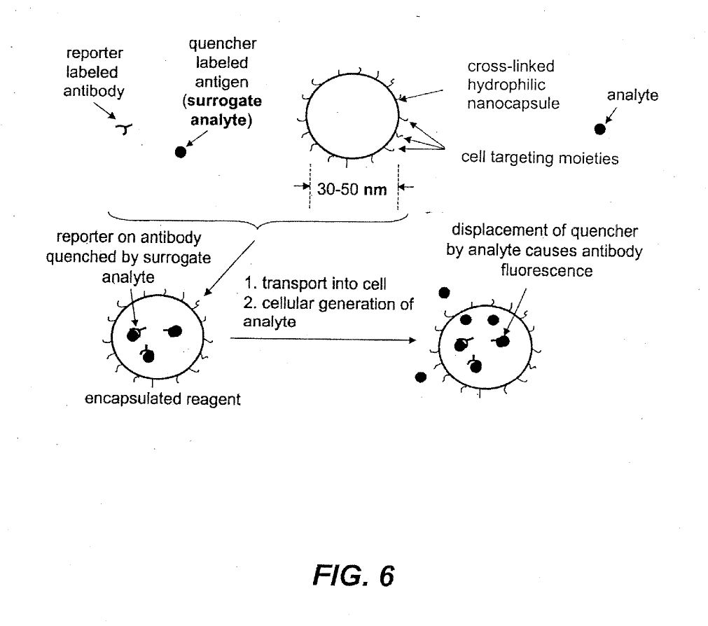

FIG. 6 illustrates an exemplary reporter system comprising one or more surrogate analyte-protein complexes, each comprising a labeled protein (“reporter labeled antibody”) and a surrogate analyte (“quencher labeled antigen”), encapsulated in a impermeable cross-linked hydrophilic nanocapsule to which can be attached targeting moieties. Suitable targeting moieties useful for introducing the nanocapsules comprising the reporter system into a cell of interest are described above. As illustrated in FIG. 6, binding of the surrogate analyte to the labeled antibody quenches the signal from the “reporter”. The “reporter” depicted in FIG. 6 can comprise any of the label moieties described herein. Passage of one or more target analytes into the capsule can displace one or more surrogate analytes, generating a measurable increase in fluorescence and indicating the presence of the target analyte.

In some embodiments, the label moiety comprises a fluorescent moiety. The fluorescent moiety can comprise any entity that provides a fluorescent signal and that can be used in accordance with the methods and principles described herein. Typically, the fluorescent moiety of the labeling molecule comprises a fluorescent dye that in turn comprises a resonance-delocalized system or aromatic ring system that absorbs light at a first wavelength and emits fluorescent light at a second wavelength in response to the absorption event. A wide variety of such fluorescent dye molecules are known in the art. For example, fluorescent dyes can be selected from any of a variety of classes of fluorescent compounds, such as xanthenes, rhodamines, fluoresceins, cyanines, phthalocyanines, squaraines, bodipy dyes, coumarins, oxazines, and carbopyronines.

In some embodiments, the fluorescent moiety comprises a xanthene dye. Generally, xanthene dyes are characterized by three main features: (1) a parent xanthene ring; (2) an exocyclic hydroxyl or amine substituent; and (3) an exocyclic oxo or imminium substituent. The exocyclic substituents are typically positioned at the C3 and C6 carbons of the parent xanthene ring, although “extended” xanthenes in which the parent xanthene ring comprises a benzo group fused to either or both of the C5/C6 and C3/C4 carbons are also known. In these extended xanthenes, the characteristic exocyclic substituents are positioned at the corresponding positions of the extended xanthene ring. Thus, as used herein, a “xanthene dye” generally comprises one of the following parent rings:

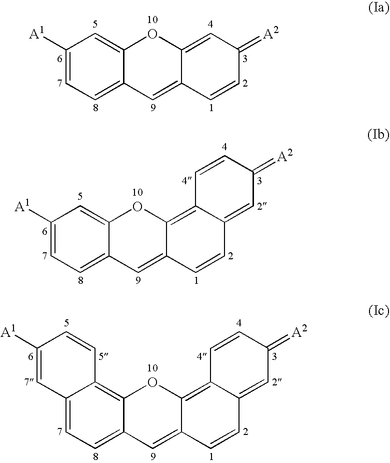

In the parent rings depicted above, A¹ is OH or NH₂ and A² is O or NH₂³⁰ . When A¹ is OH and A² is O, the parent ring is a fluorescein-type xanthene ring. When A¹ is NH₂ and A² is NH₂⁺, the parent ring is a rhodamine-type xanthene ring. When A¹ is NH₂ and A² is O, the parent ring is a rhodol-type xanthene ring.

One or both of nitrogens of A¹ and A² (when present) and/or one or more of the carbon atoms at positions C1, C2, C2″, C4, C4″, C5, C5″, C7″, C7 and C8 can be independently substituted with a wide variety of the same or different substituents. In one embodiment, typical substituents comprise, but are not limited to, —X, —R^{a}, —OR^{a}, —SR^{a}, —NR^{a}R^{a}, perhalo (C₁-C₆) alkyl, —CX₃, —CF₃, —CN, —OCN, —SCN, —NCO, —NCS, —NO, —NO₂, —N₃, —S(O)₂O, —S(O)₂OH, —S(O)₂R^{a}, —C(O)R, —C(O)X, —C(S)R^{a}, —C(S)X, —C(O)OR^{a}, —C(O)O⁻, —C(S)OR^{a}, —C(O)SR^{a}, —C(S)SR^{a}, —C(O)NR^{a}R^{a}, —C(S)NR^{a}R^{a} and —C(NR)NR^{a}R^{a}, where each X is independently a halogen (preferably —F or —Cl) and each R^{a} is independently hydrogen, (C₁-C₆) alkyl, (C₁-C₆) alkanyl, (C₁-C₆) alkenyl, (C₁-C₆) alkynyl, (C₅-C₂₀) aryl, (C₆-C₂₆) arylalkyl, (C₅-C₂₀) arylaryl, 5-20 membered heteroaryl, 6-26 membered heteroarylalkyl, 5-20 membered heteroaryl-heteroaryl, carboxyl, acetyl, sulfonyl, sulfinyl, sulfone, phosphate, or phosphonate. Generally, substituents which do not tend to completely quench the fluorescence of the parent ring are preferred, but in some embodiments quenching substituents may be desirable. Substituents that tend to quench fluorescence of parent xanthene rings comprise heavy atoms, such as —NO₂, —Br and —I, and/or other functional moieties, such as NO₂.

The C1 and C2 substituents and/or the C7 and C8 substituents can be taken together to form substituted or unsubstituted buta[1,3]dieno or (C₅-C₂₀) aryleno bridges. For purposes of illustration, exemplary parent xanthene rings including unsubstituted benzo bridges fused to the C1/C2 and C7/C8 carbons are illustrated below:

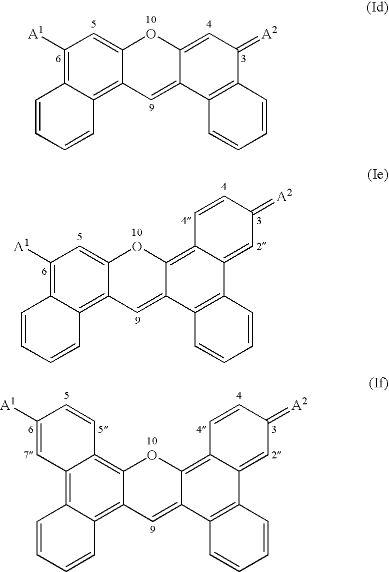

The benzo or aryleno bridges may be substituted at one or more positions with a variety of different substituent groups, such as the substituent groups previously described above for carbons C1-C8 in structures (Ia)-(Ic), supra. In embodiments including a plurality of substituents, the substituents may all be the same, or some or all of the substituents can differ from one another.

When A¹ is NH₂ and/or A² is NH₂⁺, the nitrogen atoms may be included in one or two bridges involving adjacent carbon atom(s). The bridging groups may be the same or different, and are typically selected from (C₁-C₁₂) alkyldiyl, (C₁-C₁₂) alkyleno, 2-12 membered heteroalkyldiyl and/or 2-12 membered heteroalkyleno bridges. Non-limiting exemplary parent rings that comprise bridges involving the exocyclic nitrogens are illustrated below:

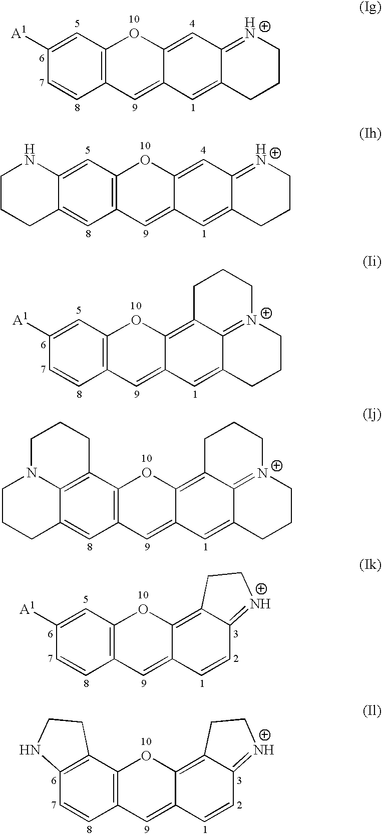

The parent ring may also comprise a substituent at the C9 position. In some embodiments the C9 substituent is selected from acetylene, lower (e.g., from 1 to 6 carbon atoms) alkanyl, lower alkenyl, cyano, aryl, phenyl, heteroaryl, and substituted forms of any of the preceding groups. In embodiments in which the parent ring comprises benzo or aryleno bridges fused to the C1/C2 and C7/C8 positions, such as, for example, rings (Id), (Ie) and (If) illustrated above, the C9 carbon is preferably unsubstituted.

In some embodiments, the C9 substituent is a substituted or unsubstituted phenyl ring such that the xanthene dye comprises one of the following structures:

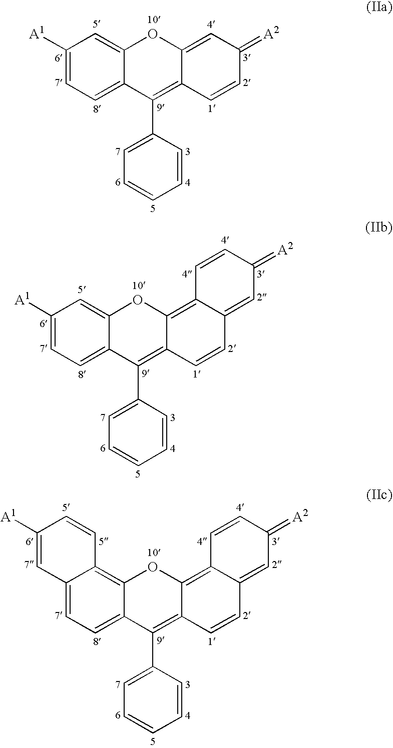

The carbons at positions 3, 4, 5, 6 and 7 may be substituted with a variety of different substituent groups, such as the substituent groups previously described for carbons C1-C8. In some embodiments, the carbon at position C3 is substituted with a carboxyl (—COOH) or sulfuric acid (—SO₃H) group, or an anion thereof. Dyes of formulae (IIa), (IIb) and (IIc) in which A¹ is OH and A² is O are referred to herein as fluorescein dyes; dyes of formulae (IIa), (IIb) and (IIc) in which A¹ is NH₂ and A² is NH₂⁺ are referred to herein as rhodamine dyes; and dyes of formulae (IIa), (Ilb) and (Ilc) in which A¹ is OH and A² is NH₂⁺ (or in which A¹ is NH₂ and A² is O) are referred to herein as rhodol dyes.

As highlighted by the above structures, when xanthene rings (or extended xanthene rings) are included in fluorescein, rhodamine and rhodol dyes, their carbon atoms are numbered differently. Specifically, their carbon atom numberings include primes. Although the above numbering systems for fluorescein, rhodamine and rhodol dyes are provided for convenience, it is to be understood that other numbering systems may be employed, and that they are not intended to be limiting. It is also to be understood that while one isomeric form of the dyes are illustrated, they may exist in other isomeric forms, including, by way of example and not limitation, other tautomeric forms or geometric forms. As a specific example, carboxy rhodamine and fluorescein dyes may exist in a lactone form.

In some embodiments, the fluorescent moiety comprises a rhodamine dye. Exemplary suitable rhodamine dyes include, but are not limited to, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dR110), tetramethyl rhodamine (TAMRA) and 4,7-dichloro-tetramethylrhodamine (dTAMRA). Additional suitable rhodamine dyes include, for example, those described in U.S. Pat. Nos. 6,248,884, 6,111,116, 6,080,852, 6,051,719, 6,025,505, 6,017,712, 5,936,087, 5,847,162, 5,840,999, 5,750,409, 5,366,860, 5,231,191, and 5,227,487; PCT Publications WO 97/36960 and WO 99/27020; Lee et al., NUCL. ACIDS RES. 20:2471-2483 (1992), Arden-Jacob, NEUE LANWELLIGE XANTHEN-FARBSTOFFE FÜR FLUORESZENZSONDEN UND FARBSTOFF LASER, Verlag Shaker, Germany (1993), Sauer et al., J. FLUORESCENCE 5:247-261 (1995), Lee et al., NUCL. ACIDS RES. 25:2816-2822 (1997), and Rosenblum et al., NUCL. ACIDS RES. 25:4500-4504 (1997). A particularly preferred subset of rhodamine dyes are 4,7-dichlororhodamines. In one embodiment, the fluorescent moiety comprises a 4,7-dichloro-orthocarboxyrhodamine dye.

In some embodiments, the fluorescent moiety comprises a fluorescein dye. Exemplary suitable fluorescein include, but are not limited to, fluorescein dyes described in U.S. Pat. Nos. 6,008,379, 5,840,999, 5,750,409, 5,654,442, 5,188,934, 5,066,580, 4,933,471, 4,481,136 and 4,439,356; PCT Publication WO 99/16832, and EPO Publication 050684. A preferred subset of fluorescein dyes are 4,7-dichlorofluoresceins. Other preferred fluorescein dyes include, but are not limited to, 5-carboxyfluorescein (5-FAM) and 6-carboxyfluorescein (6-FAM). In one embodiment, the fluorescein moiety comprises a 4,7-dichloro-orthocarboxyfluorescein dye.

In some embodiments, the fluorescent moiety can include a cyanine, a phthalocyanine, a squaraine, or a bodipy dye, such as those described in the following references and the references cited therein: U.S. Pat. Nos. 6,080,868, 6,005,113, 5,945,526, 5,863,753, 5,863,727, 5,800,996, and 5,436,134; and PCT Publication WO 96/04405.

In some embodiments, the fluorescent moiety can comprise a network of dyes that operate cooperatively with one another such as, for example by FRET or another mechanism, to provide large Stoke's shifts. Such dye networks typically comprise a fluorescence donor moiety and a fluorescence acceptor moiety, and may comprise additional moieties that act as both fluorescence acceptors and donors. The fluorescence donor and acceptor moieties can comprise any of the previously described dyes, provided that dyes are selected that can act cooperatively with one another. In a specific embodiment, the fluorescent moiety comprises a fluorescence donor moiety which comprises a fluorescein dye and a fluorescence acceptor moiety which comprises a fluorescein or rhodamine dye. Non-limiting examples of suitable dye pairs or networks are described in U.S. Pat. Nos. 6,399,392, 6,232,075, 5,863,727, and 5,800,996.

In some embodiments, the label moiety comprises a quenching moiety. The quenching moiety can be any moiety capable of quenching the fluorescence of a fluorescent moiety when it is in close proximity thereto, such as, for example, by orbital overlap (formation of a ground state dark complex), collisional quenching, FRET, or another mechanism or combination of mechanisms. The quenching moiety can itself be fluorescent, or it can be non-fluorescent. In some embodiments, the quenching moiety comprises a fluorescent dye that has an absorbance spectrum that sufficiently overlaps the emissions spectrum of a fluorescent moiety such that it quenches the fluorescence of the fluorescent moiety when in close proximity thereto.

The assays typically comprise contacting a reporter system with a sample comprising one or more target analytes of interest. In embodiments employing two or more target analytes, each labeled protein comprising the reporter system can be the same, or some, or all of the labeled proteins can differ.

The assays taught herein typically comprise the use of a buffer, such as a buffer described in the “Biological Buffers” section of the 2003 Sigma-Aldrich Catalog. Exemplary buffers include sodium phosphate, sodium acetate, PBS, MES, MOPS, HEPES, Tris (Trizma), bicine, TAPS, CAPS, and the like. The buffer is present in an amount sufficient to generate and maintain a desired pH and/or ionic strength. The pH of the binding buffer can be selected according to the pH dependency of the binding activity. For example, the pH can be from 2 to 12, from 4 to 11, or from 6 to 10. The buffer may also contain any necessary cofactors or agents required for binding. The identities and concentration of such cofactors and/or agents will depend upon the particular assay system and will be apparent to those of skill in the art. The concentration of the labeled proteins present in a reporter system may vary substantially. For example, the assay buffer can comprise from about 10⁻¹⁰ to 10⁻³ labeled proteins. In some embodiments, the assay buffer comprises from about 1 pM to 1 μM labeled proteins. If a plurality of different types of labeled proteins are used, each may comprise in the assay buffer in the above concentration ranges.

The assays typically do not require the presence of detergents or other components. In general, it is desirable to avoid high concentrations of components in the reaction mixture that can adversely affect the fluorescence properties of the reaction product, or that can interfere with the detection of target analytes.

The fluorescence signal can be monitored using conventional methods and instruments. For example, the surrogate analyte-protein complexes of the present teachings can be used in a continuous monitoring phase, in real time, to allow the user to rapidly determine whether an analyte is present in the sample, and optionally, the amount or activity of the analyte. In some embodiments, the fluorescence signal can be measured from at least two different time points. In some embodiments, the signal can be monitored continuously or at several selected time points. Alternatively, the fluorescence signal can be measured in an end-point embodiment in which a signal is measured after a certain amount of time, and the signal is compared against a control signal (sample without analyte), threshold signal, or standard curve.

The amount of the fluorescence signal generated is not critical and can vary over a broad range. The only requirement is that the fluorescence be measurable by the detection system being used. In some embodiments, a fluorescence signal that is at least 2-fold greater than the background signal can be generated upon dissociation of the surrogate analyte-protein complex. In some embodiments, a fluorescence signal that is at least 3-fold greater than the background signal can be generated upon dissociation of the surrogate analyte-protein complex. In some embodiments, a fluorescence signal that is at least 4-fold greater than the background signal can be generated upon dissociation of the surrogate analyte-protein complex. In some embodiments, a fluorescence signal that is at least 5-fold greater than the background signal can be generated upon dissociation of the surrogate analyte-protein complex. In some embodiments, a fluorescence signal between 2 to 10-fold greater than the background signal can be generated upon dissociation of the surrogate analyte-protein complex.

In some embodiments, the cross-linked hydrophilic nanocapsules can be used to encapsulate water-soluble agents. Examples of agents that can be encapsulated in the nanocapsules described herein include the therapeutic agents and diagnostic agents described in U.S. patent application publication no. 2006/0159738, the disclosure of which is incorporated herein by reference in its entirety.

### 6. EXAMPLES

Aspects of the present teachings may be further understood in light of the following examples, which should not be construed as limiting the scope of the present teachings in anyway.

6.1 Preparation of an Exemplary Cross-Linked Hydrophilic Nanocapsule

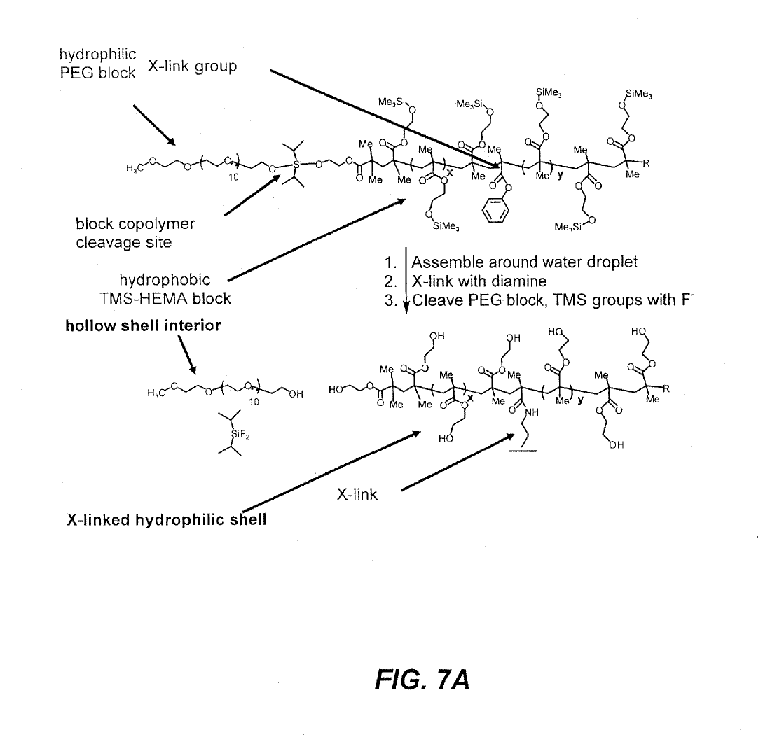

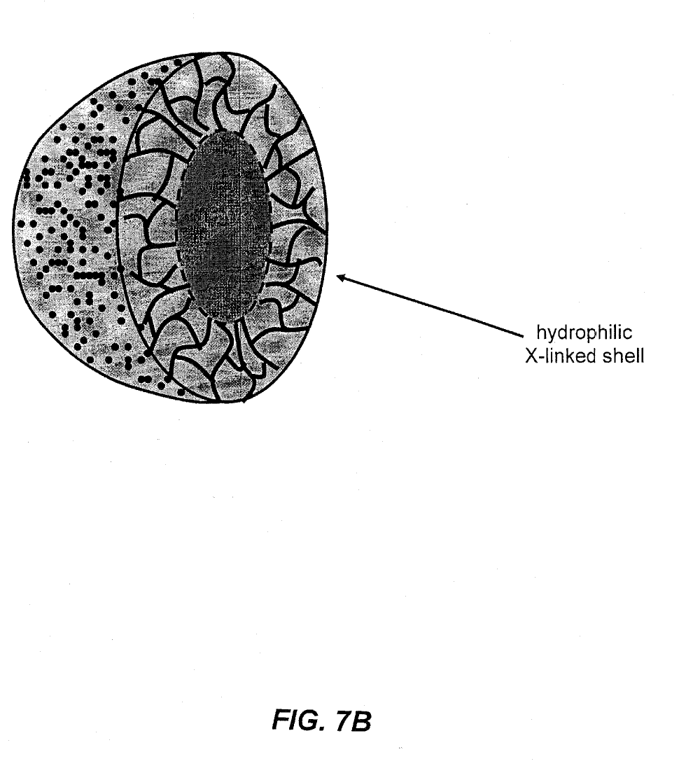

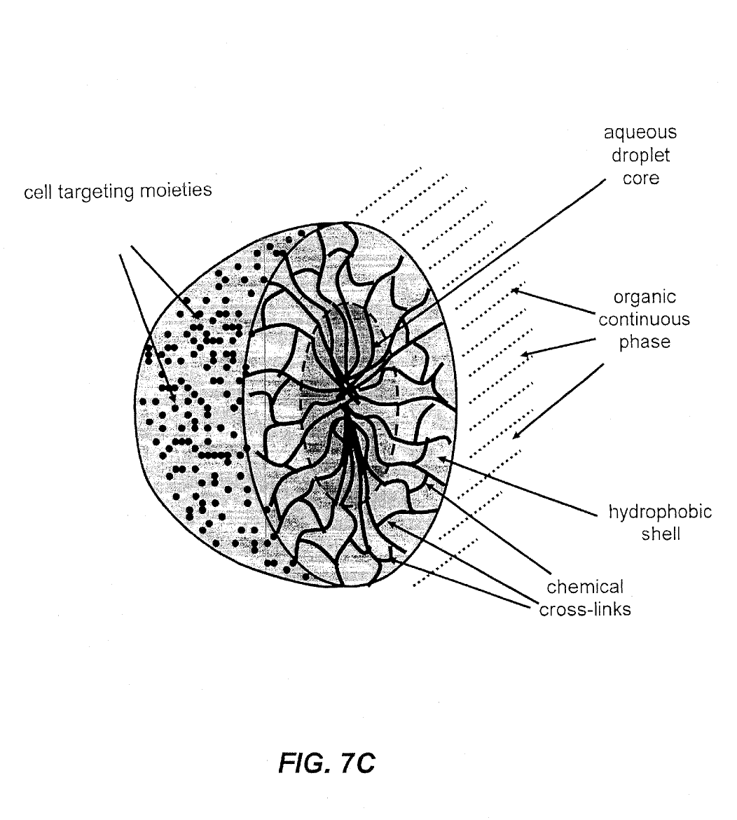

Referring to FIG. 7A, polymeric amphiphiles useful in the methods and compositions described herein can be synthesized from hydrophilic and hydrophobic polymer blocks that can be connected to one another through a cleavable linker moiety. In the exemplary embodiment illustrated in FIG. 7A, the terminus of each of the hydrophobic and hydrophilic segments includes a hydroxyl functionality, that can be connected with a dialkyl silyl group, such as diisopropyl-dichlorosilane, and subsequently cleaved using for example, a fluoride ion.

As illustrated in FIG. 7A, a significant percentage of the backbone of the hydrophobic polymer block can comprise trimethylsilyl (TMS) ethers of the polymerizable monomer hydroxyethyl methacrylate (HEMA), which imparts hydrophobic character to the TMS-HEMA polymers.

Included in the hydrophobic polymer block, are two or more monomers comprising functional groups that can be used to cross-link the polymers to each other. During polymerization, phenyl methacrylate can be added as a cross-linking agent. The amount of phenyl methacrylate added is adjusted to yield at least two phenyl methacrylate moieties per hydrophobic block. The resulting phenyl ester is reactive with an amine, such as a diamine, triamine, or the like, which, when added cross-links the monomers comprising the cross-linking functional groups to each other forming the shell of the reverse micelle.

The functional groups imparting hydrophobicity to the hydrophobic block can be removed or modified using a chemical or physical conversion process to impart a hydrophilic or water soluble character to the hydrophobic block. For example, the hydrophobic block depicted in FIG. 7A, can be treated with fluoride ion to remove the TMS protecting groups, resulting in a nanocapsule comprising a cross-linked hydrophilic shell as depicted in FIG. 7B.

As depicted in FIG. 7C, cross-linked hydrophilic nanocapsules encapsulating one or more water soluble reporter systems or agents can be formed by suspending the amphiphilic polymers in an organic suspension of aqueous droplets comprising the water soluble reporter systems. The amphiphilic polymers assemble around the aqueous droplets creating a reverse polymeric micelle comprising a hydrophobic shell and a hydrophilic core comprising the water soluble reporter system(s). Addition of a cross-linking agent results in the formation of a cross-linked hydrophobic shell. Removal or modification of functional groups imparting hydrophobicity to the hydrophobic block creates a cross-linked hydrophilic shell.

In some embodiments, the core hydrophilic block can be cleaved leaving a hollow sphere comprising a water soluble reporter system or agent.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A reverse micelle comprising:
a plurality of amphiphilic polymers, wherein each polymer, independently from the others, comprises the structure: 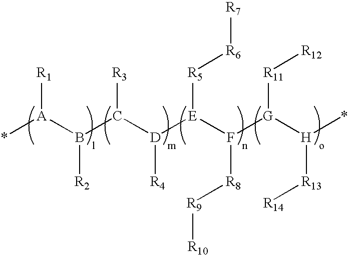
wherein
(A—B)ₗ represents a monomer unit comprising a first polymer block moiety;
[—(E—F)ₙ—(G—H)ₒ—] each independently of the other, represents monomer units comprising a second polymer block moiety;
(C—D)ₘ represents a linker moiety;
R₁, R₂, R₃, R4 represent optional substituents;
R₅—R₆—R₇ represent a first functional group, R₈—R₉—R₁₀ represent a second functional group, R₁₁—R₁₂ represent a third fuinctional group, and R₁₃—R₁₄ represent a fourth fuinctional group; and,
l and n represent integers from 4-20, m represents an integer from 0 to 1, and o represents an integer from 2 to 6.

2. A reverse micelle according to claim 1 in which (A—B) comprises a water soluble monomer unit capable of imparting water solubility to said first block.

3. A reverse micelle according to claim 2 in which (A—B) comprises a monomer unit selected from the group consisting of —O—CH₂—CH₂, —NH—CH₂—CH₂ and —S(O)—CH₂CH₂.

4. A reverse micelle according to claim 1 in which (A—B) comprises a monomer unit comprising one or more of the optional substituents R₁, and/or R₂, wherein (A—B) comprises a water insoluble monomer unit, and R₁, and/or R₂ comprise substituents capable of imparting water solubility to said first block.

5. A reverse micelle according to claim 4 in which (A—B) comprises the monomer unit —CH₂—CH₂—and R₁, and/or R₂ are selected from the group consisting of —C(O)NH₂, —C(O)OH, —C(O)O⁻, and —SO₃⁻.

6. A reverse micelle according to claim 1 in which (E—F) comprises a water soluble monomer unit comprising at least a first functional group comprising R₅—R₆—R₇ and/or a second functional group comprising R₈—R₉—R₁₀, wherein at least the first and/or the second functional group comprises one or more substituents capable of imparting water insolubility to said second block.

7. A reverse micelle according to claim 6 in which at least the first or the second functional group is cleavable to yield a water soluble monomer unit comprising (E—F) and either the first or the second functional group.

8. A reverse micelle according to claim 6 in which both the first and the second functional groups are cleavable to yield a water soluble monomer unit comprising (E—F).

9. A reverse micelle according to claim 7 in which the R₇ and/or the R₁₀ substituent is cleavable using a cleavage agent selected from the group consisting of hydroxide, acid, fluoride, and amine.

10. A reverse micelle according to claim 7 in which the R₇ and/or the R₁₀ substituent is cleavable using an enzymatic cleavage agent.

11. A reverse micelle according to claim 7 in which the R₇ and/or the R₁₀ substituent is cleavable using light.

12. A reverse micelle according to claim 1 in which (E—F) comprises a water insoluble monomer unit comprising at least a first functional group comprising R₅—R₆—R₇ and/or a second functional group comprising R₈—R₉—R₁₀, wherein at least one of the first and/or the second functional group comprises one or more substituents capable of imparting water insolubility to said second block.

13. A reverse micelle according to claim 12 in which R₇ and/or R₁₀ comprise substituents capable of imparting water insolubility to said second block.

14. A reverse micelle according to claim 14 in which the R₇ and/or the R₁₀ substituent is cleavable to yield the water insoluble monomer unit (E—F) comprising at least one water soluble functional group comprising R₅—R₆, and/or R₈—R₉.

15. A reverse micelle according to claim 13 in which the R₇ and/or the R₁₀ substituent is cleavable using a cleavage agent selected from the group consisting of hydroxide, acid, fluoride, and amine.

16. A reverse micelle according to claim 13 in which the R₇ and/or the R₁₀ substituent is cleavable using an enzymatic cleavage agent.

17. A reverse micelle according to claim 13 in which the R₇ and/or the R₁₀ substituent is cleavable using light.

18. A reverse micelle according to claim 1 in which the R₅ and/or the R₈ substituents are capable of promoting polymerization of the (E—F) monomer units.

19. A reverse micelle according to claim 6 in which the R₆ substituent is a linkage moiety capable of linking the R₅ substituent to the R₇ substituent to form the first functional group comprising R₅—R₆—R₇.

20. A reverse micelle according to claim 6 in which the R₉ substituent is a linkage moiety capable of linking the R₈ substituent to the R₁₀ substituent to form the second functional group comprising R₈—R₉—R₁₀.

21. A reverse micelle according to claim 6 in which the R₇ and/or the R₁₀ substituents are capable of imparting water insolubility to said second block.

22. A reverse micelle according to claim 6 in which at least one of R₅, R₆, R₇, R₈, R₉, and/or R₁₀ is not hydrogen.

23. A reverse micelle according to claim 6 in which at least one of the (E—F) monomer units comprises —CH(R₅—R₆—R₇)—CH(R₈—R₉—R₁₀)—, wherein R₅ and/or R₈ is carbonyl, R₆ and/or R₉ is selected from oxygen and nitrogen, and R₇ and/or R₁₀ is selected from an alkyl containing from 4 to 20 carbon atoms, phenyl, benzyl or trialkylsilyl.

24. A reverse micelle according to claim 6 in which at least one of the (E—F) monomer units comprises —CH(R₅—R₆—R₇)—CH(R₈—R₉—R₁₀)—, wherein R₅ and/or R₈ is oxygen, R₆ and/or R₉ comprises carbonyl, and R₇ and/or R₁₀ is selected from an alkoxy containing from 3 to 10 carbon atoms, phenoxy, benzoxy and trialkylsiloxy.

25. A reverse micelle according to claim 6 in which at least one of the (E—F) monomer units comprises —CH(R₅—R₆—R₇)—CH(R₈—R₉—R₁₀)—, wherein R₅ and/or R₈ is selected from nitrogen or an amine, R₆ and/or R₉ is carbonyl, and R₇ and/or R₁₀ is selected from an alkoxy containing from 3 to 10 carbon atoms, phenoxy, benzoxy and trialkylsiloxy.

26. A reverse micelle according to claim 6 in which at least one of the (E—F) monomer units comprises —CH(R₅—R₇)—CH(R₈—R₁₀)—, wherein R₅ and/or R₈ is selected from sulfate, phosphate or borate, and R₇ and/or R₁₀ is selected from an alkyl containing from 4 to 20 carbon atoms, phenyl, benzyl or trialkylsilyl.

27. A reverse micelle according to claim 6 in which at least one of the (E—F) monomer units comprises —CH₂—CH₂—N(R₅—R₇)—, wherein R₅ is carbonyl, and R₇ is selected from an alkoxy containing from 3 to 10 carbon atoms, phenoxy, benzoxy and trialkylsiloxy.

28. A reverse micelle according to claim 6 in which at least one of the (E—F) monomer units comprises —CH₂—CH—N(R₅—R₇)—, wherein R₅ is carbonyl, and R₇ is selected from an alkoxy containing from 3 to 10 carbon atoms, phenoxy, benzoxy and trialkylsiloxy.

29. A reverse micelle according to claim 6 in which at least one of the (E—F) monomer units comprises —CH₂—CH—O(R₅—R₇)—, wherein R₅ is carbonyl, and R₇ is selected from an alkoxy containing from 3 to 10 carbon atoms, phenoxy, benzoxy and trialkylsiloxy.

30. A reverse micelle according to claim 1 in which (G—H) comprises a monomer unit comprising at least a third functional group comprising R₁₁—R₁₂ and/or a second functional group comprising R₁₃—R₁₄, wherein at least one of the first and/or the second functional group comprises one or more substituents capable of reacting with a cross-linking agent.

31. A reverse micelle according to claim 30 in which one or more of the substituents comprises an electrophilic group capable of reacting with a nucleophilic cross-linking agent.

32. A reverse micelle according to claim 30 in which one or more of the substituents comprises a nucleophilic group capable of reacting with an electrophilic cross-linking agent.

33. A reverse micelle according to claim 30 in which one or more of the substituents comprises a latent anionic group capable of reacting with a multivalent metal cation.

34. A reverse micelle according to claim 30 in which the R₁₁ and/or the R₁₃ substituents are capable of promoting co-polymerization of the (G—H) monomer units with (E—F) monomer units.

35. A reverse micelle according to claim 34 in which the R₁₁ and/or the R₁₃ substituents are capable of promoting co-polymerization of the (G—H) monomer units with the (E—F) monomer units, and of reacting with a cross-linking agent.

36. A reverse micelle according to claim 30 in which the R₁₂ and/or the R₁₄ substituents are capable of reacting with a cross-linking agent.

37. A reverse micelle according to claim 30 in which at least one of R₁₁, R₁₂, R₁₃ and/or R₁₄ is not hydrogen.

38. A reverse micelle according to claim 31 in which at least one of the (G—H) monomer units comprises —CH(R₁₁—R₁₂)—CH(R₁₃—R₁₄)—, wherein R₁₁ and/or R₁₃ is selected from carbonyl, sulfate, phosphate, or borate, and R₁₂ and/or R₁₄ is an electrophilic substituent selected from alkoxy, phenoxy, substituted phenoxy, halogen, and N-hydroxysuccinimidyl, and organic or inorganic mixed anhydrides, such as acetate or phosphate.

39. A reverse micelle according to claim 31 in which at least one of the (G—H) monomer units comprises —CH(R₁₁—R₁₂)—CH(R₁₃—R₁₄)—, wherein R₁₁ and/or R₁₃ is selected from carbonyl, sulfate, phosphate, or borate, and R₁₂ and/or R₁₄ is an electrophilic substituent comprising a Michael acceptor such as vinyl.

40. A reverse micelle according to claim 31 in which at least one of the (G—H) monomer units comprises —CH(R₁₁—R₁₂)—CH(R₁₃—R₁₄)—, wherein R₁ and/or R₁₃ is selected from oxygen or amine, and R₁₂ and/or R₁₄ is an electrophilic substituent selected from alkoxycarbonyl, phenoxycarbonyl, and halocarbonyl.

41. A reverse micelle according to claim 31 in which at least one of the (G—H) monomer units comprises —CH(R₁₁—R₁₂)—CH(R₁₃—R₁₄)—, wherein R₁ and/or R₁₃ is selected from oxygen or amine, and R₁₂ and/or R₁₄ is an electrophilic substituent comprising a Michael acceptor selected from maleimide, vinylcarbonyl, alkynylcarbonyl, vinylsulfone and alkynylsulfone.

42. A reverse micelle according to claim 32 in which at least one of the (G—H) monomer units comprises —CH(R₁₁—R₁₂)—CH(R₁₃—R₁₄)—, wherein R₁ and/or R₁₃ is carbonyl, and R₁₂ and/or R₁₄ is a nucleophilic substituent selected from alcohol, polyol, amine, polyamine, sulfide or polysulfide.

43. A reverse micelle according to claim 1 in which the linker moiety (C—D) further comprises optional substituents R₃ and/or R₄.

44. A reverse micelle according to claim 1 in which (C—D) represents a linker moiety capable of being cleaved by a chemical agent, wherein said cleavage results in the removal of the first polymer block moiety.

45. A reverse micelle according to claim 1 in which (C—D) represents a linker moiety capable of being cleaved by a physical agent, wherein said cleavage results in the removal of the first polymer block moiety.

46. A cross-linked hydrophilic nanocapsule comprising: a water soluble reporter system, wherein the nanocapsule is impermeable to the diffusion of the reporter system out of the nanocapsule, and a plurality of hydrophilic polymers.

47. A cross-linked hydrophilic nanocapsule according to claim 46, wherein each polymer, independently from the others, comprises the structure: 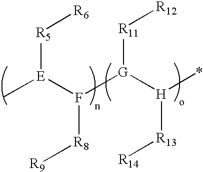
wherein
—(E—F)ₙ—(G—H)ₒ— each independently of the other, represents monomer units comprising a polymer block moiety;
R₅—R₆ represent a first water soluble functional group, R₈—R₉ represent a second water soluble functional group, R₁₁—R₁₂ represent a third functional group, and R₁₃—R₁₄ represent a fourth functional group; and
n represents an integer from 4-20 and o represents an integer from 2 to 6.

48. A cross-linked nanocapsule according to claim 47, wherein each polymer, independently from the others, comprises the structure:
—(E—F)ₙ—(G—H)ₒ—,
wherein —(E—F)ₙ—(G—H)ₒ—each independently of the other, represent monomer units comprising a hydrophilic polymer block moiety; and n represents an integer from 4-20 and o represents an integer from 2 to 6.

49. An amphiphilic polymer comprising the structure: 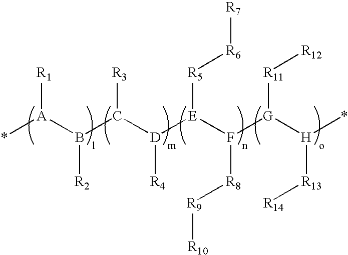
wherein
(A—B)ₗ represents a monomer unit comprising a first polymer block moiety;
[—(E—F)ₙ—(G—H)ₒ—] each independently of the other, represents monomer units comprising a second polymer block moiety;
(C—D)ₘ represents a linker moiety;
R₁, R₂, R₃, R₄ represent optional substituents;
R₅—R₆—R₇ represent a first functional group, R₈—R₉—R₁₀ represent a second functional group, R₁₁ —R₁₂ represent a third functional group, and R₁₃—R₁₄ represent a fourth functional group; and,
l and n represent integers from 4-20, m represents an integer from 0 to 1, and o represents an integer from 2 to 6.

50. An amphiphilic polymer according to claim 49 in which (A—B) comprises a water soluble monomer unit capable of imparting water solubility to said first block.

51. A method of making a reverse micelle, comprising:
emulsifying an aqueous phase with an organic phase to yield a water-in-oil emulsion, wherein the emulsion comprises a plurality of amphiphilic polymers according to claim 49.

52. A method of making a reverse micelle according to claim 51, in which the water-in-oil emulsion further comprises one or more water soluble reporter systems.

53. A method of making a reverse micelle according to claim 51, further comprising the step of adding an agent capable of cross-linking the amphiphilic polymers to each other via two or more (G—H) moieties, wherein the (G—H) moieties are located on adjacent amphiphilic polymers, wherein said cross-linking step creates a cross-linked reverse micelle.

54. A method for detecting the presence or absence of a target/analyte molecule in a sample, comprising contacting the sample with one or more cross-linked hydrophilic nanocapsules comprising an encapsulated water soluble reporter system according to claim 46, wherein said system is capable of generating a detectable fluorescent signal upon the binding of a target analyte molecule to said system that is at least 3-fold greater than the background signal.
